SEQUENCE LISTING (5 sequences)

SEQ ID NO: 1 - protein, 500 aa, Artificial Sequence
  Synthetic mature 500 aa protein
SAAATQAVPAPNQQPEVFCNQIFINNEWHDAVSRKTFPTVNPSTGEVICQVAEGDKEDVDKAVKAARAAFQLGSPWRRMDASHRGRLLNRLADLIERDRTYLAALETLDNGKPYVISYLVDLDMVLKCLRYYAGWADKYHGKTIPIDGDFFSYTRHEPVGVCGQIIPWNFPLLMQAWKLGPALATGNVVVMKVAEQTPLTALYVANLIKEAGFPPGVVNIVPGFGPTAGAAIASHEDVDKVAFTGSTEIGRVIQVAAGSSNLKRVTLELGGKSPNIIMSDADMDWAVEQAHFALFFNQGQCCCAGSRTFVQEDIYDEFVERSVARAKSRVVGNPFDSKTEQGPQVDETQFKKILGYINTGKQEGAKLLCGGGIAADRGYFIQPTVFGDVQDGMTIAKEEIFGPVMQILKFKTIEEVVGRANNSTYGLAAAVFTKDLDKANYLSQALQAGTVWVNCYDVFGAQSPFGGYKMSGSGRELGEYGLQAYTEVKTVTVKVPQKNS

SEQ ID NO: 2 - DNA, 1503 bp, Artificial Sequence
  Synthetic nucleotide sequence encoding mature 500 aa protein
tcagccgccgccacccaggccgtgcctgcccccaaccagcagcccgaggtcttctgcaaccagattttcataaacaatgaatggcacgatgccgtcagcaggaaaacattccccaccgtcaatccgtccactggagaggtcatctgtcaggtagctgaaggggacaaggaagatgtggacaaggcagtgaaggccgcccgggccgccttccagctgggctcaccttggcgccgcatggacgcatcacacaggggccggctgctgaaccgcctggccgatctgatcgagcgggaccggacctacctggcggccttggagaccctggacaatggcaagccctatgtcatctcctacctggtggatttggacatggtcctcaaatgtctccggtattatgccggctgggctgataagtaccacgggaaaaccatccccattgacggagacttcttcagctacacacgccatgaacctgtgggggtgtgcgggcagatcattccgtggaatttcccgctcctgatgcaagcatggaagctgggcccagccttggcaactggaaacgtggttgtgatgaaggtagctgagcagacacccctcaccgccctctatgtggccaacctgatcaaggaggctggctttccccctggtgtggtcaacattgtgcctggatttggccccacggctggggccgccattgcctcccatgaggatgtggacaaagtggcattcacaggctccactgagattggccgcgtaatccaggttgctgctgggagcagcaacctcaagagagtgaccttggagctgggggggaagagccccaacatcatcatgtcagatgccgatatggattgggccgtggaacaggcccacttcgccctgttcttcaaccagggccagtgctgctgtgccggctcccggaccttcgtgcaggaggacatctatgatgagtttgtggagcggagcgttgcccgggccaagtctcgggtggtcgggaacccctttgatagcaagaccgagcaggggccgcaggtggatgaaactcagtttaagaagatcctcggctacatcaacacggggaagcaagagggggcgaagctgctgtgtggtgggggcattgctgctgaccgtggttacttcatccagcccactgtgtttggagatgtgcaggatggcatgaccatcgccaaggaggagatcttcgggccagtgatgcagatcctgaagttcaagaccatagaggaggttgttgggagagccaacaattccacgtacgggctggccgcagctgtcttcacaaaggatttggacaaggccaattacctgtcccaggccctccaggcgggcactgtgtgggtcaactgctatgatgtgtttggagcccagtcaccctttggtggctacaagatgtcggggagtggccgggagttgggcgagtacgggctgcaggcatacactgaagtgaaaactgtcacagtcaaagtgcctcagaagaactcataa

SEQ ID NO: 3 - protein, 517 aa, Artificial Sequence
  Synthetic immature 517 aa protein including signal sequence
MLRAAARFGPRLGRRLLSAAATQAVPAPNQQPEVFCNQIFINNEWHDAVSRKTFPTVNPSTGEVICQVAEGDKEDVDKAVKAARAAFQLGSPWRRMDASHRGRLLNRLADLIERDRTYLAALETLDNGKPYVISYLVDLDMVLKCLRYYAGWADKYHGKTIPIDGDFFSYTRHEPVGVCGQIIPWNFPLLMQAWKLGPALATGNVVVMKVAEQTPLTALYVANLIKEAGFPPGVVNIVPGFGPTAGAAIASHEDVDKVAFTGSTEIGRVIQVAAGSSNLKRVTLELGGKSPNIIMSDADMDWAVEQAHFALFFNQGQCCCAGSRTFVQEDIYDEFVERSVARAKSRVVGNPFDSKTEQGPQVDETQFKKILGYINTGKQEGAKLLCGGGIAADRGYFIQPTVFGDVQDGMTIAKEEIFGPVMQILKFKTIEEVVGRANNSTYGLAAAVFTKDLDKANYLSQALQAGTVWVNCYDVFGAQSPFGGYKMSGSGRELGEYGLQAYTEVKTVTVKVPQKNS

SEQ ID NO: 4 - DNA, 1554 bp, Artificial Sequence
  Synthetic nucleic acid encoding immature 517 aa protein including signal sequence
atgttgcgcgctgccgcccgcttcgggccccgcctgggccgccgcctcttgtcagccgccgccacccaggccgtgcctgcccccaaccagcagcccgaggtcttctgcaaccagattttcataaacaatgaatggcacgatgccgtcagcaggaaaacattccccaccgtcaatccgtccactggagaggtcatctgtcaggtagctgaaggggacaaggaagatgtggacaaggcagtgaaggccgcccgggccgccttccagctgggctcaccttggcgccgcatggacgcatcacacaggggccggctgctgaaccgcctggccgatctgatcgagcgggaccggacctacctggcggccttggagaccctggacaatggcaagccctatgtcatctcctacctggtggatttggacatggtcctcaaatgtctccggtattatgccggctgggctgataagtaccacgggaaaaccatccccattgacggagacttcttcagctacacacgccatgaacctgtgggggtgtgcgggcagatcattccgtggaatttcccgctcctgatgcaagcatggaagctgggcccagccttggcaactggaaacgtggttgtgatgaaggtagctgagcagacacccctcaccgccctctatgtggccaacctgatcaaggaggctggctttccccctggtgtggtcaacattgtgcctggatttggccccacggctggggccgccattgcctcccatgaggatgtggacaaagtggcattcacaggctccactgagattggccgcgtaatccaggttgctgctgggagcagcaacctcaagagagtgaccttggagctgggggggaagagccccaacatcatcatgtcagatgccgatatggattgggccgtggaacaggcccacttcgccctgttcttcaaccagggccagtgctgctgtgccggctcccggaccttcgtgcaggaggacatctatgatgagtttgtggagcggagcgttgcccgggccaagtctcgggtggtcgggaacccctttgatagcaagaccgagcaggggccgcaggtggatgaaactcagtttaagaagatcctcggctacatcaacacggggaagcaagagggggcgaagctgctgtgtggtgggggcattgctgctgaccgtggttacttcatccagcccactgtgtttggagatgtgcaggatggcatgaccatcgccaaggaggagatcttcgggccagtgatgcagatcctgaagttcaagaccatagaggaggttgttgggagagccaacaattccacgtacgggctggccgcagctgtcttcacaaaggatttggacaaggccaattacctgtcccaggccctccaggcgggcactgtgtgggtcaactgctatgatgtgtttggagcccagtcaccctttggtggctacaagatgtcggggagtggccgggagttgggcgagtacgggctgcaggcatacactgaagtgaaaactgtcacagtcaaagtgcctcagaagaactcataa

SEQ ID NO: 5 - protein, 17 aa, Artificial Sequence
  Synthetic Signal peptide by itself
MLRAAARFGPRLGRRLL

(19) United States
(12) Patent Application Publication
(10) Pub. No.: US 2019/0160187 A1
(43) Pub. Date: May 30, 2019

(54) GENE THERAPY FOR THE TREATMENT OF ALDEHYDE DEHYDROGENASE DEFICIENCY

(71) Applicants: Cornell University, Ithaca, NY (US); Adverum Biotechnologies, Inc., Menlo Park, CA (US)

(72) Inventors: Mehdi Gasmi, Menlo Park, CA (US); Ronald G. Crystal, New York, NY (US); Odelya E. Pagovich, New York, NY (US)

(73) Assignees: Cornell University, Ithaca, NY (US); Adverum Biotechnologies, Inc., Menlo Park, CA (US)

(21) Appl. No.: 16/321,023

(22) PCT Filed: Jul. 26, 2017
(86) PCT No.: PCT/US2017/043999

§ 371 Date: Jan. 26, 2019

Related U.S. Application Data
(60) Provisional application No. 62/367,012, filed on Jul. 26, 2016.

(51) Int. Cl. A61K 48/00; A61P 35/00; A61P 19/10; A61P 7/06; A61P 9/10; A61P 9/06; A61P 9/12; A61P 25/28; A61K 9/00

(52) U.S. Cl. CPC A61K 48/0066; A61K 48/0058; A61K 48/0091; A61P 35/00; A61K 48/0083; A61P 19/10; C12Y 102/01003; A61P 9/10; A61P 9/06; A61P 9/12; A61P 25/28; A61K 9/0019; A61K 9/0053; A61P 7/06

(57) ABSTRACT

A vector comprising a promoter operably linked to a nucleic acid sequence encoding human aldehyde dehydrogenase, as well as a composition comprising the vector and method of using the vector to treat aldehyde dehydrogenase deficiency, or to prevent or treat a disease characterized by aldehyde dehydrogenase deficiency.

### CROSS-REFERENCE TO RELATED APPLICATIONS

This patent application claims the benefit of U.S. Provisional Patent Application No. 62/367,012, filed Jul. 26, 2016 which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 13,873 Byte ASCII (Text) file named “725826 _ST25.TXT,” created on Jul. 26, 2016.

### BACKGROUND OF THE INVENTION

Aldehyde dehydrogenases (ALDHs) belong to a superfamily of enzymes that play a key role in the metabolism of aldehydes of both endogenous and exogenous sources. Nineteen functional ALDH genes have been identified in the human genome that possess physiological and toxicological functions (Edenberg H J, Alcohol Res Health 2007, 30(1): 5-13; Steinmetz C G et al., Structure 1997, 15:5(5):701-11). Aldehyde dehydrogenase 2 (ALDH2), a key enzyme that oxidizes acetaldehyde, is crucial for alcohol metabolism. Genetic polymorphisms of human ALDH2 have been well studied among a wide range of ethnic groups (Eriksson C J, Alcohol Clin Exp Res 2001, 15S-32S; Yoshida et al., Proc. Natl. Acad. Sci 1984, 81(1):258-261). The most relevant ALDH2 variant is the ALDH2*2 allele, which is found in approximately 35-45% of East Asians (Yoshida et al., Proc. Natl. Acad. Sci 1984, 81(1):258-261; Li H et al., Ann Hum Genet 2009, 73:335-345). Approximately 560 million (8%) of the world population has this mutation making ALDH2*2 the most common human enzyme deficiency, exceeding other well-known human enzymopathies and hemoglobin disorders (Brooks P J et al., PLoS Med 2009, 6(3):e50; Chen C H et al., Physiol Rev 2014, 94(1):1-34). A variety of studies have linked ALDH2 dysfunction to multiple human diseases including aerodigestive tract cancers, cardiovascular diseases, diabetes and neurodegenerative diseases (Mandel S et al., Ann NY Acad Sci 2005, 1053:356-375; Kamino K et al., Biochem. Biophys. Res. Commun 2000, 273:192-96; Wang B et al., J. Neurol. Sci 2008, 268:172-75; Murata C et al., Alcohol. Clin. Exp Res 2000,24:5S-11S; Xu F et al., Hypertens. Res 2010, 33:49-55; Yokoyama A et al., Cancer Epidemiol. Biomarkers Prev 1996, 5:99-102; Oze I et al., Jpn. J. Clin. Oncol 2011, 41:677-92; Takagi S et al., Hypertens. Res 2002,25:677-81; Jo S A et al., Clin. Chim. Acta 2007,382:43-47; Xu F et al., J. Cell. Mol. Med 2011, 15:1955-62; Takeuchi F et al., Eur. J. Hum. Genet 2012,20:333-40; Wang Q et al., DNA Cell Biol 2013, 32:393-99; Asakage T et al., Carcinogenesis 2007, 28:865-874; Ding J H, et al., World J Gastroenterol 2009,15:2395-2400; Cui R et al., Gastroenterology 2009, 137:1768-75; Li Y et al., J Clin Invest 2006, 116:506-511; Chen Z et al., Proc Natl Acad Sci 2005, 102:12159-12164; Mackenzie I S et al., Arterioscler. Thromb. Vasc. Biol 2005, 25:1891-95; Kato N et al., Nat Genet 2011, 43:531-538; Chen C H et al., Cardiovasc Res 2010, 88(1):51-7).

The alcohol flushing syndrome characterized by facial flushing, headaches, nausea, dizziness, and cardiac palpitations after consumption of alcoholic beverages commonly observed in East Asian populations is caused by acetaldehyde accumulation manifesting as a result of reduced ALDH2 activity (Eriksson et al., Clin. Exp. Res., 2001 15S-32S). This ethanol-induced syndrome in ALDH2*2 individuals is caused by a G-to-A point mutation in exon 12 of the ALDH2 gene. This mutation results in a glutamic acid-to-lysine substitution at position 487 (E487K) in the human ALDH2 protein (Yoshida et al., Proc. Natl. Acad. Sci., 1984,81(1):258-261). The common E478K (glutamic acid to lysine) genetic polymorphism in the ALDH2 gene can result in a substantial decrease in its acetaldehyde metabolizing capacity (Yoshida et al., Proc. Natl. Acad. Sci 1984, 81(1):258-261; Baan R et al., Lancet Oncol. 2007, 8(4):292-293). Heterozygous individuals have less than 50% of the wild-type's enzymatic activity, and ALDH2*2 homozygotes have <1-4% of the wild-type activity (Farres et al., J. Biol. Chem., 1994, 269(19):13854-13860).

In addition to the Asian flush reaction, there is a definitive link between ALDH2 enzyme deficiency and upper aerodigestive tract cancers, including cancer of the oral cavity, pharynx, larynx and esophagus (Asakage T et al., Carcinogenesis 2007, 28:865-874; Ding J H, et al., World J Gastroenterol 2009, 15:2395-2400; Hashibe M et al., Cancer Epidemiol Biomarkers Prev 2006, 15(4):696-703). ALDH2-deficient individuals are at a much higher risk of esophageal cancer (specifically, squamous cell carcinoma) from alcohol consumption than individuals with fully active ALDH2 (Yokoyama A et al., Cancer Epidemiol. Biomarkers Prev 1996, 5:99-102; Oze I et al., Jpn. J Clin. Oncol 2011, 41:677-92; Ding J H, et al., World J Gastroenterol 2009, 15:2395-2400; Cui R et al., Gastroenterology 2009, 137:1768-75; Hashibe M et al., Cancer Epidemiol Biomarkers Prev 2006, 15(4):696-703). The risk for these cancers is significantly heightened by the combination of drinking alcohol and cigarette smoking (Lee C H et al., Int J Cancer 2009, 125:1134-1142; Morita M et al., Int J Clin Oncol 2010, 15:126-134). ALDH2 is the most common genetic polymorphism involved in aerodigestive malignancies and ALDH2*2 carriers are the youngest patients with esophageal cancer. There is a 7 to 12 fold increase of esophageal cancer risk in both alcoholic and nonalcoholic ALDH2*2 carrier drinkers compared to their respective wild-type ALDH2 controls (Yokoyama A et al., Cancer Epidemiol. Biomarkers Prev 1996, 5:99-102). Cigarette smoke is also a source of acetaldehyde, and individuals with the ALDH2*2 genotype who combine heavy drinking with cigarette smoking carry the greatest cancer risk (Lee C H et al., Int J Cancer 2009, 125:1134-1142; Morita M et al., Int J Clin Oncol 2010, 15:126-134). When given the same amount of ethanol, acetaldehyde concentration is two times higher in smokers than in nonsmokers. Furthermore, with concomitant cigarette smoking and ethanol exposure, smokers have a seven times higher saliva acetaldehyde concentration compared with nonsmokers (Salaspuro V et al., Int J Cancer 2004, 111:480-483). Heavy ethanol consumption and smoking in subjects carrying the ALDH2*2 genotype contribute to one of the highest known cancer risks (odds ratio of 50.1) and up to a 25-yr earlier onset of carcinoma (45 vs 70 yr old) compared with nonsmoker, nondrinker wild-type ALDH2 subjects (Brooks P J et al., PLoS Med 2009, 6(3):e50; Lee C H et al., Int J Cancer 2009, 125:1134-1142; Morita M et al., Int J Clin Oncol 2010, 15:126-134).

Therefore, there is a need to develop compositions and methods to increase ALDH2 activity and treat diseases associated with ALDH2 deficiencies. This invention provides such compositions and methods. This and other advantages of the invention will become apparent from the detailed description provided herein.

### BRIEF SUMMARY OF THE INVENTION

The invention provides a vector comprising a promoter operably linked to a nucleic acid sequence that encodes human aldehyde dehydrogenase. The invention also provides a composition comprising the vector and a method of using the vector to treat aldehyde dehydrogenase deficiency in a mammal, or treat or prevent a disease characterized by aldehyde dehydrogenase deficiency or any symptom thereof in a mammal.

### BRIEF DESCRIPTION OF THE SEVERAL VIEWS OF THE DRAWINGS

FIG. 1A is a schematic of the AAVrh.10hC1EI vector, which depicts the AAV2 inverted terminal repeats (ITR), encapsidation signal (Ψ), CMV enhancer/chicken beta-actin (CAG) promoter, optimized human ALDH2 (hALDH2) cDNA, hemagglutinin (HA) tag, and rabbit β-globulin polyadenylation signal.

FIG. 1B is a Western blot image which depicts expression of hALDH2 encoded by the AAV-hALDH2 plasmid in HEK 293T cells.

FIG. 1C is a Western blot image which depicts hALDH2 tetramer formation of hALDH2 encoded by the AAVrh.10hALDH2 vector in HEK 293T-orf6 cells.

FIG. 2A is a graph illustrating the long term in vivo mRNA expression of hALDH2 following a single intravenous administration of the AAVrh.10hALDH2 vector to C57B1/6 mice (n=4/group).

FIG. 2B is a graph illustrating the long term in vivo protein expression of hALDH2 following a single intravenous administration of the AAVrh.10hALDH2 vector to C57B1/6 mice (n=4/group).

FIG. 3 depicts the effect of treatment of ALDH2*2 mice with AAVrh.10hALDH2. Homozygous ALDH2*2 mice (n=2, 1 M/1F/group) were intravenously administered 10¹¹ gc AAVrh.10-hALDH2 or 10¹¹ gc AAVrh.10-GFP (no therapy control). Two weeks after vector administration, mice were challenged with 4 g/kg ethanol in water by intragastric gavage, and the amount of time on the balance beam before falling (max 60 sec) was measured prior to- and 24 hours post- ethanol challenge. Each dot represents an individual mouse.

### DETAILED DESCRIPTION OF THE INVENTION

The invention provides a vector which comprises, consists essentially of, or consists of a promoter operably linked to a nucleic acid sequence that encodes human aldehyde dehydrogenase. When the inventive vector consists essentially of a promoter operably linked to a nucleic acid sequence that encodes human aldehyde dehydrogenase, additional components can be included that do not materially affect the vector (e.g., genetic elements such as poly(A) sequences or restriction enzyme sites that facilitate manipulation of the vector in vitro). When the vector consists of a promoter operably linked to a nucleic acid sequence that encodes human aldehyde dehydrogenase, the vector does not comprise any additional components (i.e., components that are not endogenous to the vector and are not required to effect expression of the nucleic acid sequence to thereby provide the protein).

The vector of the invention can comprise, consist essentially of, or consist of any gene transfer vector known in the art together with the nucleic acid sequence encoding human aldehyde dehydrogenase. Examples of such vectors include adeno-associated viral (AAV) vectors, adenoviral vectors, lentiviral vectors, retroviral vectors, and plasmids. In a preferred embodiment the vector is an AAV vector.

Adeno-associated virus is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the inverted terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61:447-57 (1990)). These proteins also regulate transcription from endogenous AAV promoters and promoters within helper viruses (see, e.g., Pereira et al., J. Virol., 71:1079-1088 (1997)). The other Rep proteins modify the function of Rep78 and Rep68. The cap genes encode the capsid proteins VP1, VP2, and VP3. The cap genes are transcribed from the p40 promoter.

The inventive AAV vector can be generated using any AAV serotype known in the art. Several AAV serotypes and over 100 AAV variants have been isolated from adenovirus stocks or from human or nonhuman primate tissues (reviewed in, e.g., Wu et al., Molecular Therapy, 14(3): 316-327 (2006)). Generally, the AAV serotypes have genomic sequences of significant homology at the nucleic acid sequence and amino acid sequence levels, such that different serotypes have an identical set of genetic functions, produce virions which are essentially physically and functionally equivalent, and replicate and assemble by practically identical mechanisms. AAV serotypes 1-5 and 7-9 are defined as “true” serotypes, in that they do not efficiently cross-react with neutralizing sera specific for all other existing and characterized serotypes. In contrast, AAV serotypes 6, 10 (also referred to as Rh10), and 11 are considered “variant” serotypes as they do not adhere to the definition of a “true” serotype. AAV serotype 2 (AAV2) has been used extensively for gene therapy applications due to its lack of pathogenicity, wide range of infectivity, and ability to establish long-term transgene expression (see, e.g., Carter, B. J., Hum. Gene Ther., 16:541-550 (2005); and Wu et al., supra). Genome sequences of various AAV serotypes and comparisons thereof are disclosed in, for example, GenBank Accession numbers U89790, J01901, AF043303, and AF085716; Chiorini et al., J. Virol., 71:6823-33 (1997); Srivastava et al., J. Virol., 45:555-64 (1983); Chiorini et al., J. Virol., 73:1309-1319 (1999); Rutledge et al., J. Virol., 72:309-319 (1998); and Wu et al., J. Virol., 74:8635-47 (2000)).

AAV rep and ITR sequences are particularly conserved across most AAV serotypes. For example, the Rep78 proteins of AAV2, AAV3A, AAV3B, AAV4, and AAV6 are reportedly about 89-93% identical (see Bantel-Schaal et al., J. Virol., 73(2):939-947 (1999)). It has been reported that AAV serotypes 2, 3A, 3B, and 6 share about 82% total nucleotide sequence identity at the genome level (Bantel-Schaal et al., supra). Moreover, the rep sequences and ITRs of many AAV serotypes are known to efficiently cross-complement (i.e., functionally substitute) corresponding sequences from other serotypes during production of AAV particles in mammalian cells.

Generally, the cap proteins, which determine the cellular tropism of the AAV particle, and related cap protein-encoding sequences, are significantly less conserved than Rep genes across different AAV serotypes. In view of the ability of Rep and ITR sequences to cross-complement corresponding sequences of other serotypes, the AAV vector can comprise a mixture of serotypes and thereby be a “chimeric” or “pseudotyped” AAV vector. A chimeric AAV vector typically comprises AAV capsid proteins derived from two or more (e.g., 2, 3, 4, etc.) different AAV serotypes. In contrast, a pseudotyped AAV vector comprises one or more ITRs of one AAV serotype packaged into a capsid of another AAV serotype. Chimeric and pseudotyped AAV vectors are further described in, for example, U.S. Pat. No. 6,723,551; Flotte, Mol. Ther., 13(1):1-2 (2006); Gao et al., J. Virol., 78:6381-6388 (2004); Gao et al., Proc. Natl. Acad. Sci. USA, 99:11854-11859 (2002); De et al., Mol. Ther., 13:67-76 (2006); and Gao et al., Mol. Ther., 13:77-87 (2006).

In one embodiment, the AAV vector is generated using an AAV that infects humans (e.g., AAV2). In a preferred embodiment, the AAV vector that infects humans is AAV8 or AAV9. Alternatively, the AAV vector is generated using an AAV that infects non-human primates, such as, for example, the great apes (e.g., chimpanzees), Old World monkeys (e.g., macaques), and New World monkeys (e.g., marmosets). Preferably, the AAV vector is generated using an AAV that infects a non-human primate pseudotyped with an AAV that infects humans. Examples of such pseudotyped AAV vectors are disclosed in, e.g., Cearley et al., Molecular Therapy, 13:528-537 (2006). In one embodiment, an AAV vector can be generated which comprises a capsid protein from an AAV isolated from a rhesus macaque pseudotyped with AAV2 inverted terminal repeats (ITRs). For instance, the inventive AAV vector can comprise a capsid protein from AAV10 (also referred to as “AAVrh.10”), which infects rhesus macaques pseudotyped with AAV2 ITRs (see, e.g., Watanabe et al., Gene Ther., 17(8):1042-1051 (2010); and Mao et al., Hum. Gene Therapy, 22:1525-1535 (2011)). In another embodiment, the inventive AAV vector is a non-naturally occurring AAV vector.

The inventive vector comprises a promoter operably linked to a nucleic acid sequence that encodes human aldehyde dehydrogenase. DNA regions are “operably linked” when they are functionally related to each other. A promoter is “operably linked” to a coding sequence if it controls the transcription of the sequence.

A “promoter” is a region of DNA that initiates transcription of a particular gene. A large number of promoters from a variety of different sources are well known in the art. Representative sources of promoters include, for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Optionally, the promoter can also comprise enhancer elements (e.g., a chimeric promoter including an enhancer).

The vector also can comprise an enhancer element. The term “enhancer element” (also referred to simply as “enhancer”) as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). The vector can comprise enhancer sequences either separate from or as part of the promoter. Promoters with combined enhancer elements are known in the art as “chimeric promoters.” Enhancers can be located upstream, within, or downstream of coding sequences. (see, e.g., Niwa et al., Gene, 108:193-199 (1991); Daly et al., Proc. Natl. Acad. Sci. U.S.A., 96:2296-2300 (1999); and Sondhi et al., Mol. Ther., 15:481-491 (2007)).

The promoter of the inventive vector can comprise, consist essentially of, or consist of any promoter known in the art, including chimeric promoters. Examples of classes of such promoters include constitutively active promoters (e.g., human beta-actin, chicken beta-actin, cytomegalovirus (CMV), and SV40), cell type specific promoters (e.g., CD19 gene promoter, CaMKIIa, and UAS), or an inducible promoter (e.g., the Tet system (U.S. Pat. Nos. 5,464,758 and 5,814,618), the Ecdysone inducible system (No et al., Proc. Natl. Acad. Sci., 93:3346-3351 (1996)), the T-REX™ system (Invitrogen, Carlsbad, Calif.), the Cre-ERT tamoxifen inducible recombinase system (Indra et al., Nuc. Acid. Res., 27:4324-4327 (1999); Nuc. Acid. Res., 28:e99 (2000); U.S. Pat. No. 7,112,715; and Kramer & Fussenegger, Methods Mol. Biol., 308:123-144 (2005)), and the LACSWITCH™ System (Stratagene, San Diego, Calif.)). In an embodiment of the invention, the promoter is a constitutively active promoter, an inducible promoter, or a cell-type specific promoter. One example of a promoter is the chicken β-actin promoter. The “chicken-β-actin promoter” (also referred to as a “CAG promoter”) comprises the CMV immediate/early enhancer, the chicken beta-actin-promoter and first exon splice donor, and the rabbit beta globin splice acceptor. In one embodiment of the invention, the nucleic acid sequence encoding the aldehyde dehydrogenase may be operably linked to a chicken β-actin promoter.

“Nucleic acid sequence” is intended to encompass a polymer of DNA or RNA, i.e., a polynucleotide, which can be single-stranded or double-stranded and which can contain non-natural or altered nucleotides. The terms “nucleic acid” and “polynucleotide” as used herein refer to a polymeric form of nucleotides of any length, either ribonucleotides (RNA) or deoxyribonucleotides (DNA). These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to, methylated and/or capped polynucleotides.

The nucleic acid sequence operably linked to the promoter may comprise any nucleic acid sequence that encodes human aldehyde dehydrogenase. The nucleic acid sequence preferably encodes human aldehyde dehydrogenase 2 (ALDH2), which may be codon optimized. Techniques for codon optimization are known in the art. The nucleic acid sequence may also encode for fusion proteins which are comprised of an active protein e.g., ALDH2 and a second moiety, usually a protein, which improves the properties (e.g., efficacy, solubility, or half-life) of the active protein. Examples of the second moiety are known in the art and include, for example, the Fc domain of an immunoglobulin and polyethylene glycol (PEG).

Aldehyde dehydrogenases are a family of enzymes that play a role in the metabolism of aldehydes of both endogenous and exogenous sources. Aldehyde dehydrogenase 2 (ALDH2) oxidizes acetaldehyde and is involved in alcohol metabolism. The human ALDH2 gene is located at chromosome 12q24 and is 44 kilobase pairs in length including 13 coding exons. ALDH2 is synthesized as a 517 amino acid precursor protein in which the 17 amino acids at the n-terminus function as a mitochondrial localization sequence that targets the precursor protein to the mitochondria. The 17 amino acid mitochondrial localization sequence is cleaved in the mitochondria leaving the 500 amino acid mature ALDH2 protein monomer which combines with other monomers to form a tetramer of identical 56 kDa subunits in the mitochondria. Examples of ALDH2 amino acid and nucleotide sequences include, for example, SEQ ID NO: 1 (mature 500 aa protein); SEQ ID NO: 2 (nucleotide sequence encoding mature 500 aa protein); SEQ ID NO: 3) (immature 517 aa protein including signal sequence; also GenBank NP_000681.2 and AAA51693.1) and SEQ ID NO: 4 (nucleic acid encoding immature 517 aa protein including signal sequence; also GenBank NM_000690.3 and AH002599.2), which nucleic acid sequences can further be codon optimized.

The nucleic acid sequence encoding the human aldehyde dehydrogenase and vector comprising same can be generated using methods known in the art. For example, nucleic acids sequences, polypeptides, and protein can be recombinantly produced using standard recombinant DNA methodology (see, e.g., Sambrook et al., Molecular Cloning: A Laboratory Manual, 3rd ed., Cold Spring Harbor Press, Cold Spring Harbor, NY, 2001; and Ausubel et al., Current Protocols in Molecular Biology, Greene Publishing Associates and John Wiley & Sons, NY, 1994). Further, a synthetically produced nucleic acid sequence encoding human aldehyde dehydrogenase can be isolated and/or purified from a source, such as bacterium, an insect, or a mammal, e.g., a rat, a human, etc. Methods of isolation and purification are well-known in the art. Alternatively, the nucleic acid sequences described herein can be commercially synthesized. In this respect, the nucleic acid sequence can be synthetic, recombinant, isolated, and/or purified. The sequences can further be optimized for increased mRNA stability and to reduce the possibility of trans-inhibition by the mutant mRNA.

In addition to the promoter operably linked to a nucleic acid sequence encoding human aldehyde dehydrogenase, the vector can comprise additional expression control sequences, such as enhancers, polyadenylation signals, transcription terminators, internal ribosome entry sites (IRES), 5′ and 3′ untranslated regions, introns, and the like, that provide for the expression of the nucleic acid sequence in a host cell. Exemplary expression control sequences are known in the art and described in, for example, Goeddel, Gene Expression Technology: Methods in Enzymology, Vol. 185, Academic Press, San Diego, Calif. (1990).

The vector may further comprise a nucleotide sequence encoding a signal peptide operably linked to the nucleic acid sequence that encodes human aldehyde dehydrogenase. When the nucleotide sequence encoding signal peptide is present, it will be located downstream of the promoter sequence such that the encoded signal peptide and aldehyde dehydrogenase are linked to one another. The vector may encode any signal peptide suitable for transport across the mitochondrial membrane (mitochondrial localization sequence), where the signal peptide is cleaved to provide the mature protein. The signal peptide should be positively charged and form a helix. In a preferred embodiment, the signal peptide is a mitochondrial localization sequence. The mitochondrial localization sequence can comprise, for instance, the amino acid sequence of SEQ ID NO: 5.

Also provided is a composition comprising, consisting essentially of, or consisting of the above-described vector and a pharmaceutically acceptable (e.g. physiologically acceptable) carrier. When the composition consists essentially of the inventive vector and a pharmaceutically acceptable carrier, additional components can be included that do not materially affect the composition (e.g., adjuvants, buffers, stabilizers, anti-inflammatory agents, solubilizers, preservatives, etc.). When the composition consists of the inventive vector and the pharmaceutically acceptable carrier, the composition does not comprise any additional components except as specified. Any suitable carrier can be used within the context of the invention, and such carriers are well known in the art. The choice of carrier will be determined, in part, by the particular site to which the composition may be administered and the particular method used to administer the composition. The composition optionally can be sterile with the exception of the vector described herein. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, Pa. (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. Preferably, the carrier is a buffered saline solution. More preferably, the inventive vector is administered in a composition formulated to protect the inventive vector from damage prior to administration and enhance transduction efficiency. For example, the composition can be formulated to reduce loss of the vector on devices used to prepare, store, or administer the vector, such as glassware, syringes, or needles. The composition can be formulated to decrease the light sensitivity and/or temperature sensitivity of the vector. To this end, the composition preferably comprises a pharmaceutically acceptable liquid carrier, such as, for example, those described above, and a stabilizing agent selected from the group consisting of polysorbate 80, L-arginine, polyvinylpyrrolidone, trehalose, and combinations thereof. Use of such a composition will extend the shelf life of the vector, facilitate administration, and increase the efficiency of the inventive method. Formulations for vector-containing compositions are further described in, for example, Wright et al., Curr. Opin. Drug Discov. Devel., 6(2):174-178 (2003) and Wright et al., Molecular Therapy, 12:171-178 (2005)).

In addition, one of ordinary skill in the art will appreciate that the inventive vector can be present in a composition with other therapeutic or biologically-active agents. For example, factors that control inflammation, such as ibuprofen or steroids, can be part of the composition to reduce swelling and inflammation associated with in vivo administration of the vector. Antibiotics, i.e., microbicides and fungicides, can be present to treat existing infection and/or reduce the risk of future infection, such as infection associated with gene transfer procedures.

The invention provides a method of treating aldehyde dehydrogenase deficiency, particularly ALDH2 deficiency, or treating or preventing a disease characterized by aldehyde dehydrogenase deficiency or any symptom thereof, in a mammal. The method comprises administering the vector described herein to the mammal, whereupon the nucleic acid is expressed to produce an aldehyde dehydrogenase protein and, thereby, treat the aldehyde dehydrogenase deficiency and/or treat or prevent the disease or symptom associated therewith.

The mammal can be any mammal with an aldehyde dehydrogenase deficiency, such as a mammal with a mutation in a gene encoding aldehyde dehydrogenase protein that results in absence of protein, a non-functional protein, or a protein with reduced function as compared to the wild-type protein. The mammal can be human, particularly a human with a deficiency in human ALDH2. In a specific embodiment, the human is heterozygous or homozygous for the ALDH2*2 allele (i.e., a glutamic acid-to-lysine substitution at position 487 (E487K) in the human ALDH2 protein, or 504 (E504K) in the immature sequence (Yoshida et al., Proc. Natl. Acad. Sci., 1984, 81(1):258-261)).

In some embodiments, the method can further comprise selecting a patient for treatment by identifying a loss-of-function mutation in a gene encoding an aldehyde dehydrogenase protein. The method can comprise, for instance, analyzing the amino acid or nucleic acid sequence encoding ALDH2 and determining whether residue 487 of the mature 500 amino acid human ALDH2 protein (or the corresponding amino acid 504 in the 517 amino acid precursor ALDH2 protein) is glutamine or some other amino acid (e.g, lysine). The subject is selected as having an ALDH2 deficiency (and a suitable candidate for treatment) when the amino acid at residue 487 of the mature ALDH2 protein (or residue 504 of the precursor ALDH2 protein) is not glutamine (i.e., has been mutated to any other amino acid, particularly lysine).

The mammal can be afflicted with a symptom or disease characterized by aldehyde dehydrogenase deficiency, particularly ALDH2 deficiency, or at risk of developing such a symptom or disease, for example, as a result of having an aldehyde dehydrogenase deficiency. Examples of diseases associated with aldehyde dehydrogenase deficiency include ethanol toxicity, upper respiratory/digestive tract cancers (e.g., cancer of the oral cavity, pharynx, larynx and esophagus), osteoporosis, radiation dermatitis, squamous cell carcinoma, fanconi anemia, diabetic complications, Parkinson's disease, Alzheimer's disease, stroke, hypertension, cardiac arrhythmia, myocardial infarction, and nitroglycerin intolerance. Symptoms of aldehyde dehydrogenase deficiency, particularly ALDH2 deficiency, may include any of those symptoms commonly associated with the foregoing diseases. For instance, symptoms of ALDH2 deficiency is the accumulation of acetaldehyde in the liver and/or blood, as well as facial flushing, headaches, nausea, dizziness, and/or cardiac palpitations after alcohol consumption.

Treating a deficiency in aldehyde dehydrogenase encompasses increasing aldehyde dehydrogenase activity or protein levels by any amount. Treating a disease characterized by a deficiency in aldehyde dehydrogenase or a symptom thereof encompasses ameliorating or slowing the progress of any physiological response or symptom brought on by the disease to any degree. Preventing a disease by a deficiency in aldehyde dehydrogenase or a symptom thereof encompasses delaying the onset of any physiological response or symptom brought on by the disease by any amount.

Any route of administration can be used to deliver the composition to the mammal. Indeed, although more than one route can be used to administer the composition, a particular route can provide a more immediate and more effective reaction than another route. Preferably, the composition is administered via intramuscular injection. A dose of composition also can be applied or instilled into body cavities, absorbed through the skin (e.g., via a transdermal patch), inhaled, ingested, topically applied to tissue, or administered parenterally via, for instance, intravenous, intraperitoneal, intraoral, intradermal, subcutaneous, or intraarterial administration.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the AAV vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the vector in the composition administered to the mammal will depend on a number of factors, including the size (mass) of the mammal, the extent of any side-effects, the particular route of administration, and the like. Preferably, the inventive method comprises administering a “therapeutically effective amount” of the composition comprising the inventive vector described herein. A “therapeutically effective amount” refers to an amount effective, at dosages and for periods of time necessary, to achieve a desired therapeutic result. The therapeutically effective amount may vary according to factors such as the degree of allergen sensitivity, age, sex, and weight of the individual, and the ability of the vector to elicit a desired response in the individual. A “prophylactically effective amount” refers to an amount effective, at dosages and for periods of time necessary, to achieve a desired prophylactic result (e.g., prevention of ALDH2 deficiency induced ethanol toxicity or upper respiratory/digestive tract cancer).

The vector encoding aldehyde dehydrogenase may be administered multiple times during a therapeutic or prophylactic treatment period and/or employ multiple administration routes, e.g., intramuscular and subcutaneous, to ensure sufficient exposure of cells to the composition. For example, the composition may be administered to the mammal two or more times (e.g., 2, 3, 4, 5, 6, 7, 8, 9, or 10 or more times) during a therapeutic or prophylactic treatment period. However, according to preferred aspects of the invention, a single administration of the vector described herein (or composition comprising the vector) is sufficient to provide a prolonged expression of the aldehyde dehydrogenase at therapeutic or prophylactic levels in the mammal. Preferably, the therapeutic levels are expressed in the mammal, after administration of the vector or composition comprising same, for about 30 days or more (e.g., about 45 days or more, about 60 days or more, about 75 days or more, about 90 days or more, about 4 months or more, about 6 months or more, about 10 months or more, or even about 12 months or more). Thus, in some embodiments, the method comprises administering the vector to the mammal not more than once within about 30 days, not more than once within about 45 days, not more than once within about 60 days, not more than once within about 75 days, or even not more than once within about 90 days (e.g., not more than once within about 4 months, about 5 months, about 6 months, about 10 months, or about 12 months).

The dose of vector in the composition required to achieve a particular therapeutic or prophylactic effect typically is administered in units of vector genome copies per cell (gc/cell) or vector genome copies/per kilogram of body weight (gc/kg). One of ordinary skill in the art can readily determine an appropriate vector dose range to treat a patient having a particular immune response based on these and other factors that are well known in the art.

The invention also comprises a method of producing the inventive AAV vector. In one embodiment the method comprises co-transfecting the AAV-ALDH2 vector along with a plasmid carrying the AAV Rep proteins derived from an AAV serotype needed for vector replication into a cell, the AAV viral structural proteins VP1, 2, and 3, that define the serotype of the produced AAV vector, and the adenovirus helper functions of E2, E4, and VA RNA. The amino acid sequence of the AAV Rep proteins and the AAV structural proteins can be from any AAV known in the art. In a preferred embodiment the AAV Rep proteins are from AAV2 and the AAV structural proteins are from AAVrh.10. The cell in which the vector and plasmid are transfected can be any cell known in the art. In a preferred embodiment the cell is an adherent cell. In a particularly preferred embodiment the cell is a human embryonic kidney (HEK) 293 cell. In an alternative embodiment the inventive AAV vector is produced in a baculovirus system.

The following examples further illustrate the invention but, of course, should not be construed as in any way limiting its scope.

### EXAMPLE 1

This example demonstrates the development of a vector comprising a promoter operably linked to a nucleic acid sequence that encodes human aldehyde dehydrogenase.

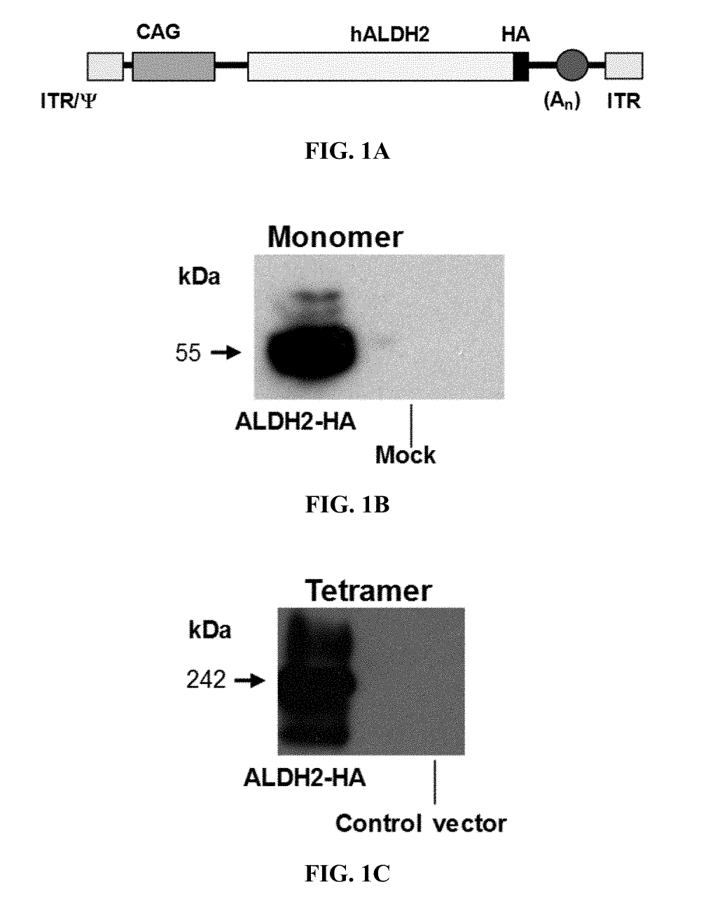

The expression cassette consists of the AAV2 inverted terminal repeats (ITR), encapsidation signal (Ψ), cytomegalovirus (CMV) enhancer chicken β-actin promoter (CAG promoter) operably linked to human ALDH2 cDNA sequence and the rabbit β-globin polyadenylation signal (FIG. 1A). The ALDH2 cDNA sequence was constructed with a c-terminal hemagglutinin (HA) tag to distinguish the ADLH2 cDNA from the mouse ALDH2 protein. The ALDH2 cDNA was optimized for increased mRNA stability and to reduce the possibility of trans-inhibition by the mutant mRNA. ALDH2 cDNA was sequence-optimized using human-biased codons and removal of: mRNA instability elements; low (<30%) or rich (>80%) GC regions; translation initiation sequences within the coding region; and potential splicing signals. Optimized ALDH2 cDNA was synthesized with an optimal Kozak consensus.

The optimized full-length ALDH2 cDNA sequence was synthesized and cloned into the pAAV plasmid-under control of the CAG promoter. The AAV-hALDH2 plasmid was produced by co-transfection into human embryonic kidney 293T cells (HEK 293T; American Type Culture Collection) of the pAAV plasmid together with a plasmid carrying the AAV Rep proteins derived from AAV2 needed for vector replication, the AAVrh.10 viral structural (Cap) proteins VP1, 2 and 3, which define the serotype of the produced AAV vector; and the adenovirus helper functions of E2, E4 and VA RNA. The AAV-hALDH2-HA vector (referred to as “AAVrh.10hALDH2”) was purified by iodixanol gradient and QHP anion exchange chromatography. Vector genome titers were determined by quantitative TaqMan real-time PCR analysis. A vector coding for an irrelevant protein was used as control for certain expression studies.

To assess AAVr.10hALDH2 directed expression of the human ALDH2 protein in vitro, HEK 293T cells were transfected with the AAV-hALDH2 plasmid or mock transfected, and supernatant was harvested 72 hours later. Human ALDH2 expression in supernatant was evaluated by SDS-PAGE and Western analysis with an anti-HA antibody. As shown in FIG. 1B, human ALDH2 was detected in cell culture supernatants. The results from this example show the expression of ALDH2 from an AAV vector.

To assess ALDH2 tetramer formation from AAVrh.10hALDH2 directed expression of the human ALDH2 protein in vitro, HEK 293T cells were infected with the AAVrh.10hALDH2 vector or a control AAVrh.10-hα1 AT vector, and supernatant was harvested 72 hours later. Human ALDH2 tetramer formation in supernatant was evaluated by SDS-PAGE and Western analysis with an anti-HA antibody. As shown in FIG. 1C, tetramers of human ALDH2 were detected in cell culture supernatants. The results from this example show that the expression of ALDH2 from an AAV vector results in the formation of ALDH2 tetramers.

### EXAMPLE 2

This example demonstrates the long term in in vivo expression of a vector comprising a promoter operably linked to a nucleic acid sequence that encodes human aldehyde dehydrogenase.

To evaluate long-term in vivo serum expression of human ALDH2 after a single treatment with the AAVrh.10hALDH2 vector, C57B1/6 male and female mice were administered the AAVrh.10hALDH2 vector, the AAVrh.10-hα1 AT vector (control vector), or phosphate buffered saline (PBS) (n=4/group) at 10¹¹ genome copies (gc) by intravenous injection in about 100μl volume. Two weeks after vector administration, total RNA and protein were isolated from liver homogenates and hALDH2 mRNA expression was analyzed by qPCR and protein expression was analyzed by Western using an anti-HA antibody.

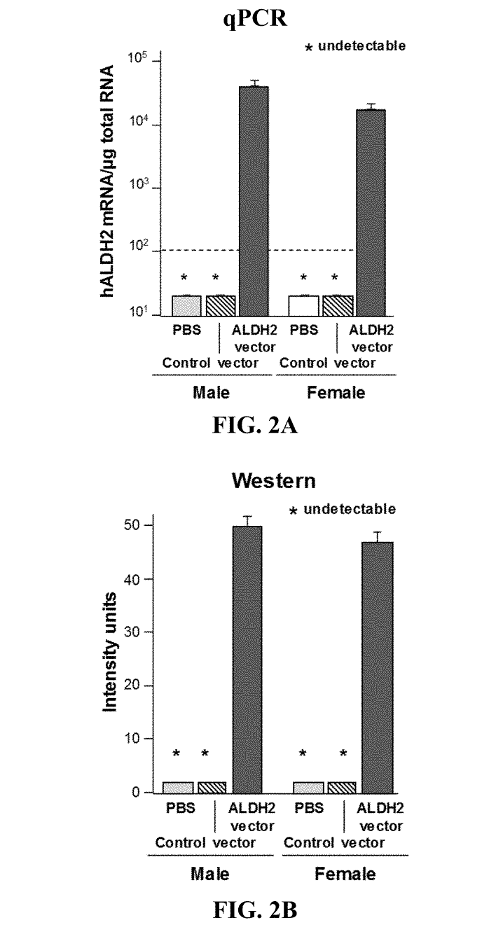

As shown in FIGS. 2A and 2B, high levels of hALDH2 mRNA (6.58 ±2.2 ×10⁴ (male) and 1.25 ±5.4 ×10⁴ (female) mRNA copies per μg of total RNA) and high levels of hALDH2 protein were detected in animals that received AAVrh.10hALDH2.

These data demonstrate that that AAV-vector mediated expression of ALDH2 provides long-term ALDH2 expression from a single administration.

### EXAMPLE 3

This example demonstrates the protection from ethanol-related toxicity by administering the AAVrh.10hALDH2 in a mouse model of ALDH2 deficiency.

To assess the in vivo activity of ALDH2 expressed from the AAVrh.10hALDH2 vector, ALDH2*2 mice with an E487K mutation equivalent to the human ALDH2*2 allele (Zambelli et al., Sci Trans Med., 6:251ra118 (2014); Jin et al., PNAS, 112:9088-9093 (2015)) were administered a single intravenous injection of AAVrh.10hALDH2 at 10¹¹ genome copies (gc) or a single intravenous injection of AAVrh.10-GFP (control) at 10¹¹ genome copies (n=2, 1male/1female/group).

Two weeks post vector administration, mice were challenged with ethanol (4 g/kg) by intragastric gavage. Six hours post-challenge, mice displayed high blood alcohol content that was reduced to near background by 24 hours (data not shown). Behavior was evaluated at 24 hours post-challenge by the balance beam test (time to fall) (Carter et al., Current Protocols Neuroscience, Chapter 8:Unit 8 (2001)).

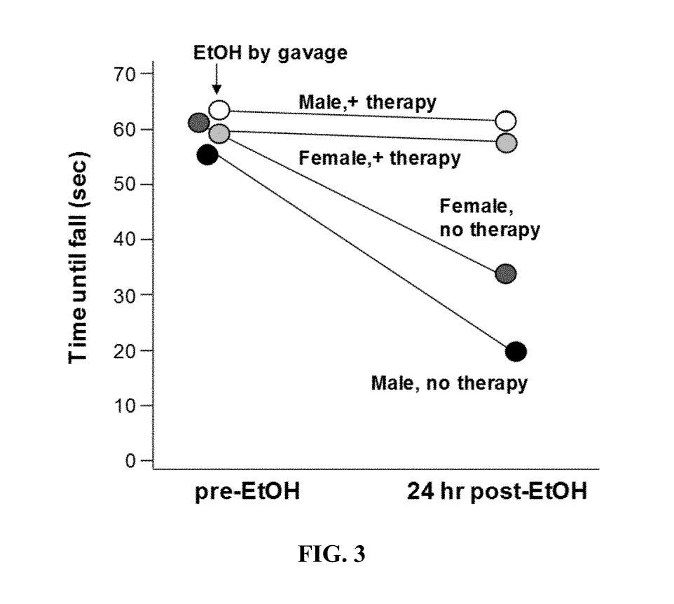

As shown in FIG. 3, the AAVrh.10hALDH2 treated male and female mice remained on the beam for the full allotted time (i.e., 60 seconds), while the mice administered the control vector fell off the beam at an earlier time.

These data demonstrate that ALDH2 expressed from the AAVrh.10hALDH2 vector is active in vivo, and can protect against ethanol-related toxicity.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A vector comprising a promoter operably linked to a nucleic acid sequence that encodes human aldehyde dehydrogenase.

2. The vector of claim 1, wherein the vector is selected from the group consisting of adeno-associated virus (AAV), adenovirus, lentivirus, retrovirus, and plasmid.

3. The vector of claim 2, wherein the vector is an AAV vector.

4. The vector of claim 3, wherein the AAV vector is a non-human adeno-associated virus.

5. The vector of claim 4, wherein the non-human adeno-associated virus is a rhesus macaque adeno-associated virus.

6. The vector of claim 5, wherein the rhesus macaque adeno-associated virus is the adeno-associated virus serotype rh.10.

7. The vector of claim 1, wherein the promoter is a constitutively active promoter.

8. The vector of claim 1, wherein the promoter is a cell type specific promoter.

9. The vector of claim 1, wherein the promoter is an inducible promoter.

10. The vector of claim 7, wherein the constitutively active promoter is a chicken beta-actin promoter.

11. The vector of claim 1, wherein the human aldehyde dehydrogenase is human aldehyde dehydrogenase 2 (ALDH2).

12. The vector of claim 1, wherein the vector further comprises a nucleic acid sequence encoding a mitochondrial localization sequence operably linked to the nucleic acid sequence that encodes human aldehyde dehydrogenase sequence.

13. The vector of claim 1, wherein the vector encodes SEQ ID NO: 1.

14. The vector of claim 12, wherein the vector encodes SEQ ID NO: 3.

15. A composition comprising the vector of claim 1 and a pharmaceutically acceptable carrier.

16. A method of treating aldehyde dehydrogenase deficiency in a mammal, or treating or preventing a disease characterized by aldehyde dehydrogenase deficiency or any symptom thereof in a mammal, the method comprising administering a vector of claim 1 to the mammal.

17. The method of claim 16, wherein the mammal is a human.

18. The method of claim 17, wherein the mammal is heterozygous or homozygous for an ALDH2*2 allele.

19. The method of claim 16, wherein the mammal is afflicted with a disease characterized by a deficiency in aldehyde dehydrogenase is selected from the group consisting of ethanol toxicity, upper respiratory/digestive tract cancer, osteoporosis, squamous cell carcinoma, fanconi anemia, Parkinson's disease, Alzheimer's disease, stroke, hypertension, cardiac arrhythmia, myocardial infarction, and nitroglycerin intolerance.

20. The method of claim 16 wherein the vector is administered to the mammal not more than once within about 30 days.

21. The method of claim 16 wherein the vector is administered by intraoral, intramuscular, transdermal, intravenous, intraarterial, subcutaneous, intradermal, or intraperitoneal administration.

22. The method of claim 16, further comprising analyzing the amino acid sequence or nucleic acid sequence of the mammal, and selecting the mammal for treatment if residue 487 of ALDH2 protein in the mammal is not glutamine.

23. The method of claim 22, wherein the subject is selected for treatment when the amino acid at residue 487 is lysine.

24-25.(canceled)
